(19) United States
(12) Patent Application Publication
(10) Pub. No.: US 2011/0319839 A1
(43) Pub. Date: Dec. 29, 2011

(54) DISPOSABLE SURGICAL INSTRUMENTS

(76) Inventor: Daniel A. Del Vecchio, Wrentham, MA (US)

(21) Appl. No.: 13/172,086

(22) Filed: Jun. 29, 2011

Related U.S. Application Data
(60) Provisional application No. 61/359,504, filed on Jun. 29, 2010.
(60) Provisional application No. 61/359,609, filed on Jun. 29, 2010.
(60) Provisional application No. 61/452,946, filed on Mar. 15, 2011.

(51) Int. Cl. A61M 5/32

(52) U.S. Cl. 604/272

(57) ABSTRACT

Various disposable cannulas and retractable cutting instruments are disclosed for use in cosmetic surgical procedures and the like.

### RELATED APPLICATIONS

This application claims the benefit of U.S. App. No. 61/359,504 filed on Jun. 29, 2010, U.S. App. No. 61/359,609 filed on Jun. 29, 2010, and U.S. App. No. 61/452,946 filed on Mar. 15, 2011. The entire content of these applications is incorporated herein by reference.

### BACKGROUND

There remains a need for improved surgical tools for use in cosmetic surgery and similar procedures.

### SUMMARY

Various disposable cannulas and retractable cutting instruments are disclosed for use in cosmetic surgical procedures and the like.

### BRIEF DESCRIPTION OF THE FIGURES

The invention and the following detailed description of certain embodiments thereof may be understood by reference to the following figures:

FIG. 1 shows a cannula with a linear reinforcement.

FIG. 2 shows a cannula with a spiral reinforcement.

FIG. 3 is a cross-section of a reinforced cannula.

FIG. 4 is a cross-section of a reinforced cannula.

FIG. 5 is a side view of an axial cutting instrument.

FIG. 6 is a top view of an axial cutting instrument.

FIG. 7 is a top view of an axial cutting instrument.

FIG. 8 shows a lateral cutting instrument in a retracted position.

FIG. 9 shows a lateral cutting instrument in a deployed position.

FIG. 10 shows an end cutting instrument in a retracted position.

FIG. 11 shows an end cutting instrument in a deployed position.

FIG. 12 shows an end cutting instrument.

FIG. 13 shows a cutting instrument.

FIG. 14 shows a handling for a cutting instrument.

FIG. 15 shows an end cutting instrument.

FIG. 16 shows an end cutting instrument.

FIG. 17 shows a side cutting instrument.

FIG. 18 shows a side cutting instrument.

FIG. 19 shows a spatula cutting instrument.

FIG. 20 shows a spatula cutting instrument.

FIG. 21 shows a spatula cutting instrument.

FIG. 22 shows a spatula cutting instrument.

FIG. 23 shows a cutting instrument.

FIG. 24 shows a cutting instrument.

FIG. 25 shows a kit for a surgical procedure.

### DETAILED DESCRIPTION

Disclosed herein are various cannulas for use in liposuction, fat grafting, and similar procedures, as well as injection or implantation in cosmetic procedures or the like. A cannula as disclosed herein may be fabricated from a clear plastic or other clear material. While such a cannula advantageously permits intraoperative monitoring of tissue being extracted from a donor site, many inexpensive clear materials suitable for use in a disposable device are also subject to cracking, fracturing, or the like. As such, a disposable cannula described herein further includes one or more reinforcing fibers that can retain the structural integrity of the cannula if a wall of the cannula cracks or fractures during use. A cannula as described herein may advantageously permit a surgeon or other use to inspect substances within the cannula for blood, clogged tissue, pathogens, and the like during use.

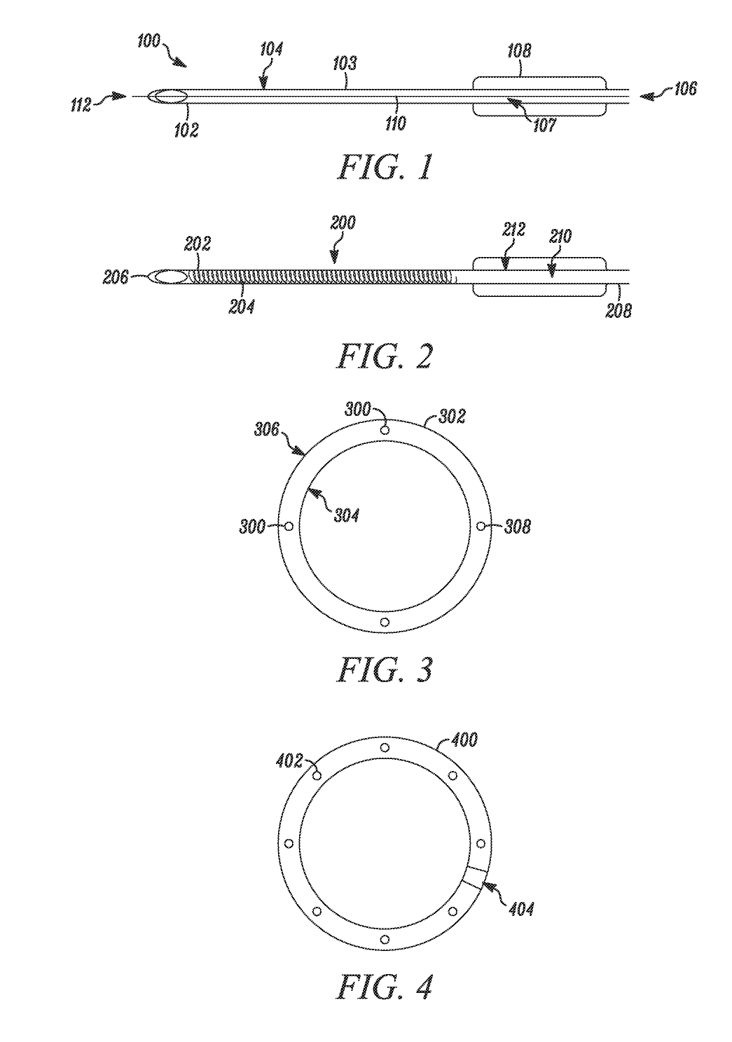

FIG. 1 shows a cannula with a linear reinforcement. In general, the cannula 100 includes an insertion tip 102, a barrel 103 with an interior channel 104, a port 106, a handle 108, and one or more reinforcing fibers 110. In general, the cannula 100 may be shaped and sized for use in a specific surgical procedure such as liposuction, fat harvesting, fat grafting, or cosmetic filling.

The insertion tip 102 may have an opening 110 coupled by the interior channel 104 to the exit 106 so that fat or other tissue and/or fluids can be extracted from a surgical site in a liposuction procedure or the like. The tip 102 may include a sharpened leading edge so that the insertion tip 102 can be inserted by an application of force through skin into a surgical site or used to cut tissue or other material within a surgical site. It will be appreciated that the term ‘sharpened’ refers to the structure of the leading edge, rather than a specific technique used to obtain that structure. Thus the tip may actually be sharpened during manufacture, or the sharpened leading edge may result from the fabrication process used to make the insertion tip 102, without any need for additional sharpening steps. In another aspect, the insertion tip 102 may included a spatulated end, e.g., an end that extends and/or flattens on one side of the barrel 103 to assist in controlled distribution of an implanted material during a filling procedure or the like. The spatulated end may be blunted in order to avoid damage to tissue or other implant structures.

The barrel 103 may usefully be formed of a transparent material to permit visual monitoring of tissue extraction during a surgical procedure. The transparent material may be any material with sufficient strength for use as a cannula 100 as contemplated herein. The transparent material may, for example, include one or more of a glass, a plastic, a polymer, an epoxy or other curable substance, a transparent ceramic, or any other light transmissive material or composite. The transparent material is preferably biocompatible, so that no further coatings or treatment are required for use in surgery. The barrel 103 may also or instead be formed of a translucent material which would still permit visual monitoring (although less detailed) of contents of the barrel 103. More generally, the barrel 103 may be fabricated of any material sufficiently clear to permit visual intraoperative monitoring of a substance in or passing through the cannula 100 (or more specifically, in or passing through the barrel 103 of the cannula 100). The barrel 103 may be formed of multiple different materials having different optical properties including at least one transparent material or at least one translucent material.

In one aspect, the barrel 103 may include clear materials and/or reinforcing fibers only at a section or sections where the barrel 103 is expected to pass from within a body to outside a body, thus providing a window visible to surgeon during a procedure. The cannula 100 or the barrel 103 may be formed of disposable materials, which may in general be any sufficiently inexpensive material for one-time use. In one aspect, the disposable materials need not be sterilizable by a surgeon after use, although they may be sterile when provided to the surgeon and may be enclosed in sterile packaging. The barrel 103 and/or insertion tip 102 may usefully incorporate a coating or impregnation of heparin, antibiotics, or other medication to improve fat flow and decrease likelihood of clotting or infection during use.

The interior channel 104 may be of any suitable diameter for extraction of fat or the like. Conventional liposuction cannulas are commercially available in standard lengths of 4, 5, 6, 7, 8, 9, 10, 12, 15, 20, 25, 30, and 35 centimeters, and outside diameters of 1.2, 1.4, 1.6, 2.1, 2.4, 3.0, 3.7, 4.6, and 6.3 millimeters. More generally, the cannula 100 may have any dimensions suitable for an intended surgical procedure. For example, a cannula for use in fat grafting or harvesting may usefully have an outside diameter of about 2 mm to about 8 mm, and a length of about 5 to about 40 cm. Cannulas are also rated by industry standard “gauges”, and a useful injection cannula for fat transplantation may have a gauge from 12 gauge to 16 gauge.

The port 106 and the insertion tip 102 may be coupled in fluid communication by an interior 107 of the barrel 103 so that fluid or other material can pass between the insertion tip 102 and the port 106 through the barrel 103. The port 106 may include a hub for connecting to suction equipment such as a 60 cc hub, a 35 cc hub, a Luer lock, or the like. It will be understood that the ‘term’ port is used here only as a term of convenience, and is not intended to imply a specific directionality to material flow through the cannula, unless such a meaning is explicitly provided or otherwise clear from the context. For example, in a cosmetic implant procedure, fat or other material may be introduced into a surgical site through the cannula. In such instances, the port may function as an entrance for fluid or other material to flow into the barrel 103 of the cannula 100. Conversely, where the surgical procedure is an extraction, the port 106 may function as an exit for withdrawing (e.g., through an application of reverse pressure) material from the barrel 103.

The handle 108 may surround the barrel 103 and provide an area where the cannula 100 can be securely gripped by a user. In addition to an exterior surface suitable for gripping, the handle 108 may protect the barrel 103 so that a user can apply a firm grip without crushing or otherwise damaging the barrel 103.

The one or more reinforcing fibers 110 may, for example be steel wires, carbon fibers, or the like, which may be arranged in lateral, circumferential, or helical patterns, or any other pattern or combination of patterns within the transparent material of the barrel 103 of the cannula 100. Other fibers such as Kevlar, or various polymers or composites may also or instead be employed to add strength to the cannula 100. It will be appreciated that the reinforcement may be structural reinforcement that resists bending or breaking of the barrel 103, or the reinforcement may flex with the barrel 103 in response to applied forces while resisting separation of fractured portions of the barrel 103 from the rest of the cannula 100, thereby maintaining structural integrity and mitigating loss of fractured portions of a cannula 100 within a surgical site. The reinforcing fibers 110 depicted in FIG. 1 are linear or straight fibers that extend directly from the insertion tip 102 to the exit 106; however it will be appreciated that different fiber arrangements may also or instead be employed. For example, the one or more reinforcing fibers 110 may be fabricated of softer polymers or the like in a mesh that bends or otherwise yields with the cannula 100 to prevent separation of pieces of the cannula 100 in the event of a fracture. Still more generally, any reinforcing mesh, reinforcing matrix, reinforcing fibers, or other weave or composite of one or more fibers may be employed as the one or more reinforcing fibers 110 to structurally support the barrel 103 of the cannula and/or contain separation of pieces of the cannula 100 in the event of a fracture. All such variations as would be apparent to one of ordinary skill are intended to fall within the scope of this disclosure.

The one or more reinforcing fibers 110 may be embedded within the cannula 110 during manufacturing, such as by casting the reinforcing fibers 110 within the sidewall or end portions of the cannula 100 during an injection molding process or the like. Similarly, the reinforcing fibers 110 may be threaded into the material during an extrusion process, or used as a wireframe or the like in a glass blowing process. More generally, any technique suitable for adding fibers or other reinforcement to a cannula during fabrication may be usefully adapted to adding the one or more reinforcing fibers 110 to the cannula 100 as contemplated herein.

The reinforcing fibers 110 may be used along an entire length of the cannula 100 or barrel 103, or some portion thereof, such as from the insertion tip 102 to the handle 108. For example, in a linear version of a reinforcing structure, the reinforcing fibers 110 may run substantially the length of the cannula 100, although it will be readily appreciated that fibers may also or instead run only a portion of the length, or may vary in length about the circumference of the cannula.

FIG. 2 shows a cannula with a spiral reinforcement. In a spiral or helical version of a reinforcing structure, the cannula 200 may have one or more fibers 202 wound about the circumference of the barrel 204 as the fibers 202 traverse the cannula 200 from one end 206 to another 208. Where multiple fibers 202 are employed, they may be parallel to one another along there length thereby forming a helical coil of two or more fibers 202. Other reinforcing structures such as square, rectangular, or triangular meshes may also or instead be employed without departing from the scope of the invention. In other embodiments, the reinforcing structure may be adhered or otherwise affixed to an interior 210 and/or exterior 212 of the cannula 204, which may simplify construction.

FIG. 3 is a cross-section of a reinforced cannula. In general one or more fibers 300 may be embedded within a barrel 302 of a cannula between an interior 304 and an exterior 306 of the barrel 302. It will be readily understood that while four fibers 300 are depicted, any number of fibers 300 may be employed. For example, the number of fibers may be 2, 4, 6, 8, 10, 12, or any other odd or even number of fibers, and the fiber density may be different at different positions long the barrel 302. The fibers 300 may be equally circumferentially spaced about the barrel 302 as depicted in the figure, or the fibers 300 may be unequally or asymmetrically spaced. In one aspect, one of the fibers 300 for structural reinforcement may be replaced or augmented with a fiber optic 308, or the barrel 302 may provide a hollow port through which the fiber optic 308 can be threaded. The fiber optic 308 may run an entire length of the barrel 302, and may include a lens or other focusing element on the insertion tip end. In this manner, a surgical site can be illuminated by supplying light to the other end of the fiber optic 308, which may include an optical coupler or other suitable component for coupling to a light source. An illumination source of this type may be advantageously used in an otherwise conventional cannula, e.g., a cannula fabricated from stainless steel or the like, that is not transparent or translucent. In another aspect, a cannula may include stainless steel portions and clear portions.

By employing linear or spiral wires or other reinforcing fibers spaced, e.g., circumferentially within and parallel to the walls of a barrel, a cannula may be provided that reduces risks of breakage within subcutaneous tissue during use, and/or maintains structural integrity after a fracture of the barrel material so that portions of the barrel are not separated within a patient's tissue.

FIG. 4 is a cross-section of a reinforced cannula. In the embodiment of FIG. 4, the barrel 400 of a cannula has eight reinforcing fibers 402 equally circumferentially spaced about the barrel 400. The fibers 402 may be straight axial fibers (per FIG. 1), helically coiled fibers (per FIG. 2), or any other suitable arrangement of fibers. The barrel 400 may include one or more holes 404 or ports from an interior of the barrel 400 to an exterior of the barrel 400 along a length of the barrel 400 from the insertion tip to the handle. In general, the holes may facilitate distribution of material during an injection or implantation material. Any number of holes 404, e.g., five holes, may be employed along the length of the barrel 400, which holes 404 may be equally or unequally spaced along the length of the barrel 400, and may be aligned in a straight line along one side of the barrel 400 or distributed on different sides/surfaces of the barrel 400 according to a desired distribution of injected material.

Also disclosed herein are embodiments of a disposable device for controlled incisions of fibrous tissue or the like, along with methods of using same in surgical procedures such as internal breast shaping after fat grafting or any similar augmentation procedure. In general, the device may include one or more cutting surfaces that may be deployed after insertion and/or rotated along an axis of the device for controlled cutting. An oversized handle may be provided for improved user control over rotational orientation of the cutting surface(s).

In general, the device may be a disposable cutting instrument, which advantageously permits the use of edge shapes and configurations that would otherwise be difficult to sterilize and/or re-sharpen. The device may for example range in size from 0.5 mm to 3 mm in external diameter.

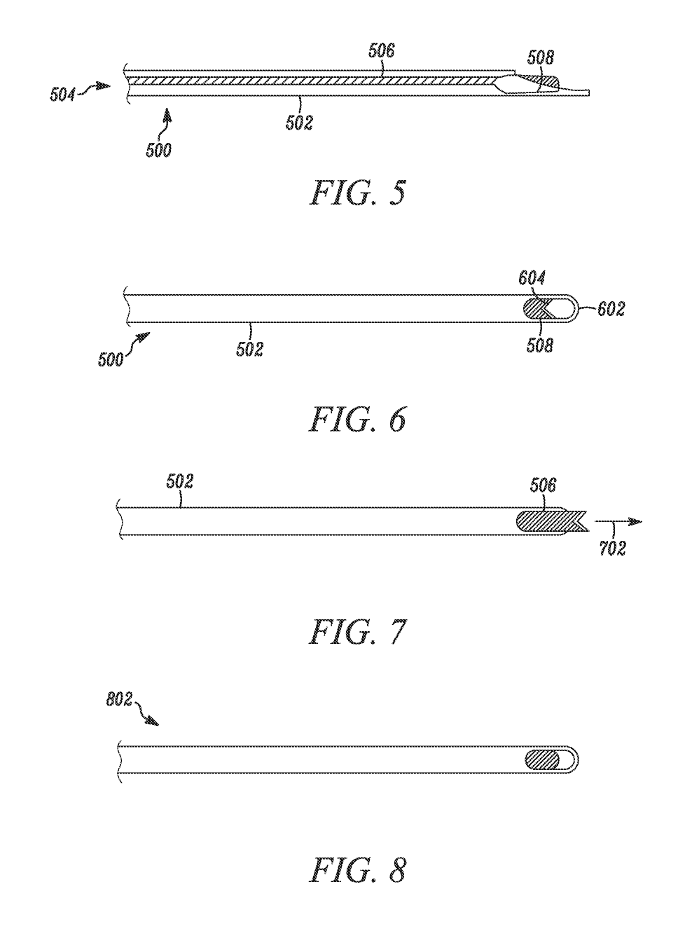

FIG. 5 is a side view of an axial cutting instrument. In general, the device 500 may include sleeve 502 with an interior 504 through which a cutting instrument 506 including a sharpened end 508 can fit.

The sleeve 502 may be formed of any biocompatible material or other material suitable for surgical instruments, including by way of example and not limitation surgical stainless steel. The sleeve 502 may also or instead be formed of a disposable material, and may be provided in a sterile packaging for use as a one-time instrument.

The interior 504 may be cylindrical, with a smoothed interior surface suitable for passage of the cutting instrument 506. More generally, the interior 504 may include any lumen for passage of an instrument therethrough.

The cutting instrument 506 may be shaped and sized to pass freely or substantially freely through the interior 504 so that a user can move the sharpened end 508 out of the sleeve 502 where the sharpened end 508 can be used in a surgical procedure (e.g., cutting tissue or other material in a cosmetic procedure). The sharpened end 508 may also be freely retractable so that it is contained within the sleeve 502 and no cutting surface is exposed. In this manner, the sleeve 502 may be conveniently positioned within a surgical site without risk of cutting or damaging surrounding tissue, and once the tip of the sleeve 502 is suitable positioned, the sharpened end 508 can be extended for use in a surgical operation.

FIG. 6 is a top view of an axial cutting instrument. As shown in FIG. 6, the device 500 of FIG. 5 may include a tip 602 that is rounded or otherwise smoothed so that the tip 602 can be freely positioned within a surgical site without cutting or otherwise disturbing surrounding tissue. The sharpened end 508 may include a notch 604 which may be any concave surface to facilitate axial cutting by extension of the tip 602 into tissue. FIG. 6 shows the sharpened end 508 in a retracted position, that is, with the sharpened end 508 contained within the tip 602 of the sleeve 502 so that the tip 602 can be freely positioned within a surgical site.

FIG. 7 is a top view of an axial cutting instrument. As explained above, the cutting instrument 506 can moved axially through the sleeve 502 (as indicated by arrow 702) into an extended position where the cutting instrument 506 can be used for surgery.

FIG. 8 shows a lateral cutting instrument in a retracted position. In general, the lateral cutting instrument 802 may be retracted and extended as described above.

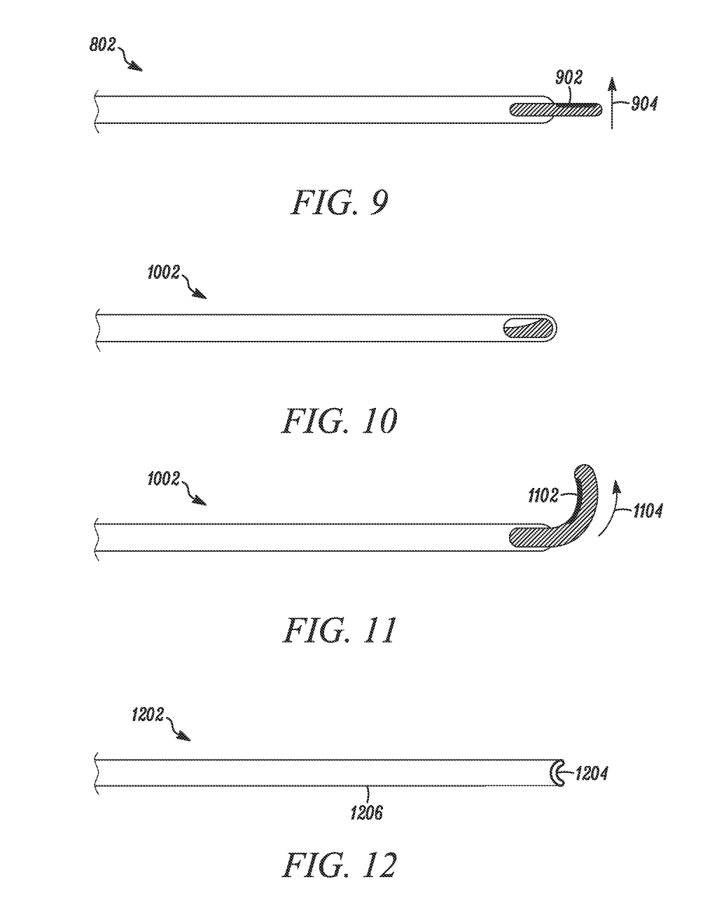

FIG. 9 shows a lateral cutting instrument in a deployed position. In the extended or deployed position, the lateral cutting instrument 802 may be used in a surgical procedure. As shown in FIG. 9, the lateral cutting instrument 802 may include a sharpened edge 902 on an edge of the cutting instrument 802, rather than on the end of the cutting instrument (as per FIG. 7). With the sharpened edge 902 so positioned, the lateral cutting instrument may be used for sideways cutting procedures as indicated by an arrow 904. It should be understood that the direction of the arrow 904 (and similar arrows throughout this specification) is not intended to require a specific direction to cutting procedures but rather to generally indicate the orientation of the cutting surface. Thus for example the lateral cutting instrument 802 may be manipulated to move in the direction of the arrow 904, or to move axially so that the sharpened edge 802 moves in a slicing or sawing motion.

FIG. 10 shows an end cutting instrument in a retracted position. In general, the end cutting instrument 1002 may be retracted and extended as described above, although the instrument 1002 may include a spring or any other mechanism to permit non-axial deployment as depicted in FIG. 11.

FIG. 11 shows an end cutting instrument in a deployed position. The end cutting instrument 1002 may include a sharpened end 1102 on an interior curved edge of the end cutting instrument 1002. So arranged, the sharpened end 1102 may be used in a pulling or scythe-like cutting motion, which may be particularly useful, e.g., for cutting ligamentous bands or other tough tissue.

FIG. 12 shows an end cutting instrument. As shown in FIG. 12, the end cutting instrument 1202 may include a sharpened end 1204 with a notch or the like fabricated directly into a sleeve 1206. In this manner, the sleeve 1206 may be used directly as a surgical cutting instrument. The sleeve 1206 may also contain an interior with another cutting instrument as described above so that, with retraction and deployment of the other cutting instrument, the end cutting instrument 1202 can also be used as a lateral cutting instrument or other type of cutting instrument by deploying the second cutting instrument through the interior of the sleeve 1206.

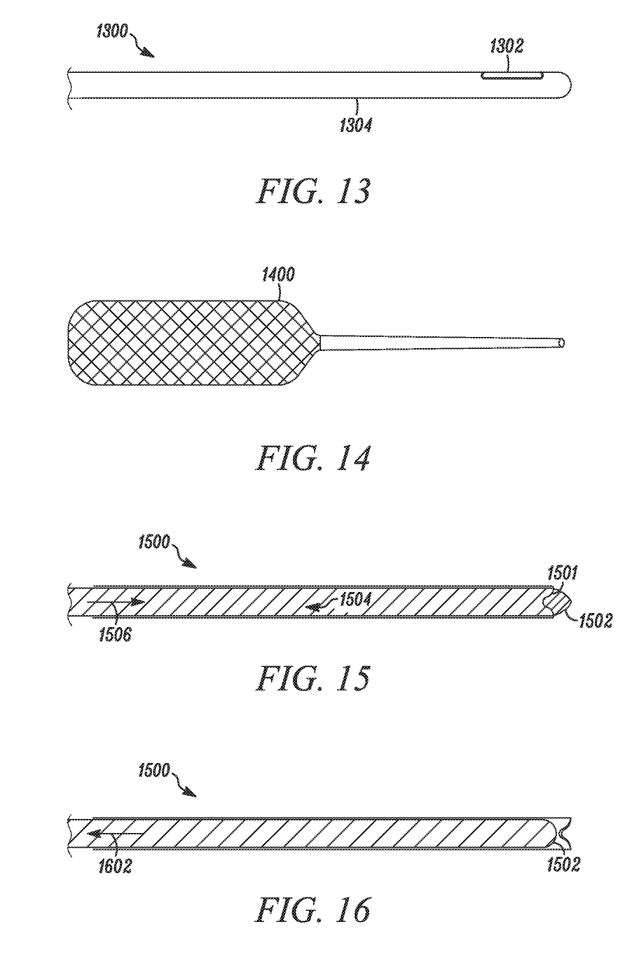

FIG. 13 shows a cutting instrument. The cutting instrument 1300 may include a sharpened end 1302 on an edge of the sleeve 1304 to accommodate lateral cutting. The cutting instrument 1300 may also include an interior for a second cutting instrument as generally described above.

FIG. 14 shows a handling for a cutting instrument. In general, the handle 1400 may have a paddle or other over-sized feature for convenient gripping and manipulation of the cutting instrument on an opposing end to the sharpened end. The handle 1400 may for example be used with any of the cutting instruments described above.

FIG. 15 shows an end cutting instrument. In general, the cutting instrument 1500 may have a sharpened end 1501 fabricated into a sleeve as described above. The cutting instrument 1500 may also include a protective tip 1502 in an interior thereof 1504, which may be extended beyond the sharpened end 1501 to prevent cutting by the sharpened end 1501 during handling or positioning of the cutting instrument 1500 at a surgical site. The protective tip 1502 may be deployed by moving the protective tip within the interior 1504 as indicated by an arrow 1506 into a deployed position where the protective tip 1502 extends beyond the sharpened end 1501 and prevents cutting by the sharpened end 1501.

FIG. 16 shows an end cutting instrument. The end cutting instrument 1500 may be as described above with reference to FIG. 15. FIG. 16 more particularly shows the end cutting instrument with the protective tip 1502 retracted by moving the protective tip 1502 as shown by an arrow 1602.

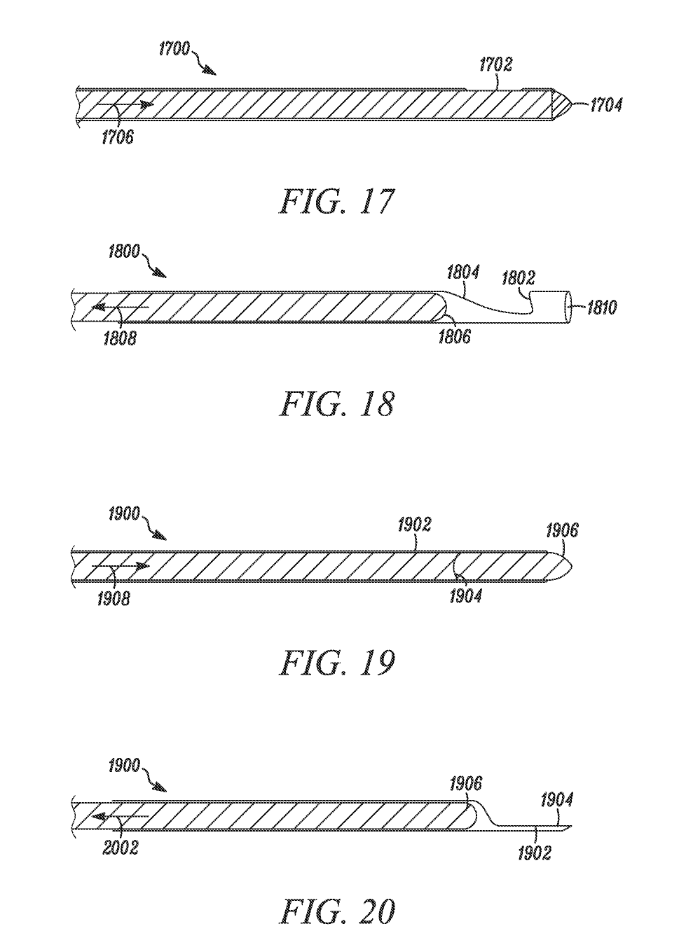

FIG. 17 shows a side cutting instrument. The side cutting instrument 1700 may include a sharpened end 1702 on a side or edge, with a protective tip 1704 reducing exposure of the sharpened end 1702 when passing through an interior of the side cutting instrument 1700 as shown. The protective tip 1704 may be deployed, e.g., by moving the protective tip 1704 as indicated by an arrow 1706.

FIG. 18 shows an end cutting instrument. The end cutting instrument 1800 may include a sharpened end 1802 on an interior curved edge of the end cutting instrument 1800. So arranged, the sharpened end 1802 may be used in a pulling or scythe-like cutting motion, which may be particularly useful, e.g., for cutting ligamentous bands or other tough tissue. The end cutting instrument 1800 may also or instead include a second sharpened end 1804 (which may be a continuation of the sharpened end 1802 or a separate sharpened portion) for lateral cutting. As depicted, a protective tip 1806 is in a retracted position, as indicated by an arrow 1808. An end 1810 of the end cutting instrument may include a cap to receive and engage the protective tip 1806, or the end 1810 may be open so that the protective tip 1806 can pass therethrough when deployed.

FIG. 19 shows a spatula cutting instrument. The spatula cutting instrument 1900 may include a flattened cutting surface 1902 with a sharpened end 1904. As depicted, a protective tip 1906 is deployed beyond the sharpened end 1904 (as shown by an arrow 1908) to prevent cutting by the sharpened end 1904.

FIG. 20 shows a spatula cutting instrument. More specifically, FIG. 20 shows a side view of the spatula cutting instrument 1900, with the protective tip 1906 in a retracted position (as shown by an arrow 2002) so that the sharpened end 1904 is exposed for use in a cutting procedure.

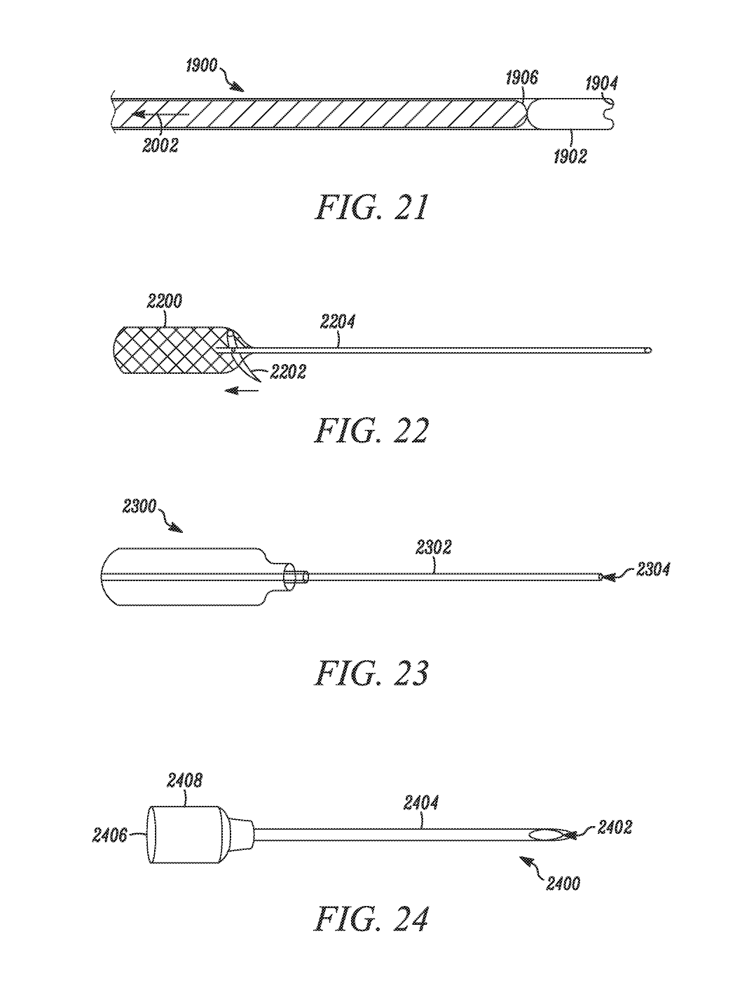

FIG. 21 shows a spatula cutting instrument. More specifically, FIG. 21 shows a top view of the spatula cutting instrument 1900, with the protective tip 1906 in a retracted position (as shown by an arrow 2002) so that the sharpened end 1904 is exposed for use in a cutting procedure.

FIG. 22 shows a spatula cutting instrument. In particular, FIG. 22 shows a handle 2200 of a cutting instrument with a control 2202, which may be finger or thumb-activated, for deploying and retracting any of the protective tips or cutting instruments described above. The control 2202 may be pivotably coupled to the handle 2200, and further coupled (e.g., pivotally) to a sleeve and a shaft 2204 of a cutting instrument or protective tip so that the tool can be deployed or retracted relative to a surrounding sleeve (not shown) by operating the control 2202 without any independent axial movement of the handle 2200. In this manner, a surgeon or other use can know that whether a cutting edge of a tool is deployed (by operation of the control 2202) independent of how the handle 2200 is manipulated to position a cutting instrument connected thereto. In general, the handle 2200 may be coupled to a sheath, and/or to a cutting instrument or protective tip within the sheath. The handle 2200 may coupled in any manner that permits control of the working end (e.g., with the sharpened end) from the handle, and that permits movement of the sheath relative to the cutting instrument or protective tip by operation of the control 2202. The control may be a finger-activated lever, as depicted, or any other suitably manipulatable control.

FIG. 23 shows a cutting instrument. The cutting instrument 2300, which may be any of the cutting instruments described above, may be autoclavable or otherwise sterilizable and/or cleanable for re-use or the cutting instrument 2300 may be disposable. The cutting instrument 2300 may be reinforced along a portion 2302 that can be positioned within the interior of a syringe tip (FIG. 24) so that the cutting instrument 2300 provides reinforcement to the syringe tip during insertion of the syringe tip into a surgical site. The portion 2302 of the cutting instrument 2300 that is reinforced (e.g., with a steel rod or other reinforcing material or structure) may be of sufficient length so that, when inserted into the syringe tip, the portion 2302 runs along a length of the interior of the syringe tip such as the entire length or substantially the entire length of the syringe tip. The portion 2302 that is reinforced may also or instead reinforce a Luer Lock junction or other connector on the non-cutting end of the syringe tip (see FIG. 24). The cutting instrument 2300 may include a sharpened end 2304 such as any of the sharpened ends described above.

FIG. 24 shows a cutting instrument. More specifically, FIG. 24 shows a syringe tip 2400 that may have an interior 2402 shaped and sized for passage of the cutting instrument 2300 of FIG. 23 therethrough. The needle 2404 of the syringe tip 2400 may be stabilized by a rigid steel rod or other reinforcement that slides into the interior 2402 of the needle 2404 during insertion of the needle 2402 into a surgical site, for example through a dermal layer into subcutaneous tissue. The syringe tip 2400 may include a Luer Lock junction or other connector 2408 on the non-cutting end 2406 to permit convenient attachment/replacement/detachment of the syringe tip, such as when the cutting edge dulls during a procedure.

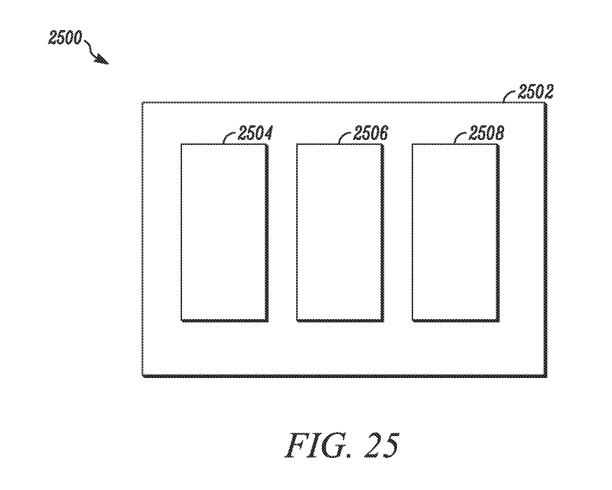

FIG. 25 shows a kit for a surgical procedure. Any of the devices described above may be provided as a kit 2500, which may, for example be contained in a sterile packaging 2502, which may include foil, plastic, and/or any other suitable material. The kit 2500 may include a number of components 2504, 2506, 2508 such as cannulas, syringe tips, cutting instruments (including, e.g., multiple cutting instruments with various sharpened ends as discussed above), connectors, various length and thickness of cannulas for injection or removal of fluids and other tissue, handles, and so forth. Thus there is disclosed herein a kit provided in a sterile packaging, the kit comprising one or more of a cannula, a syringe tip, and a cutting instrument, such as any of the components described above.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

1. A device comprising:
a cannula including a barrel with an insertion tip and a port coupled in fluid communication by an interior of the barrel, wherein the barrel is formed of a material that is sufficiently clear to permit intraoperative monitoring of a substance passing through the cannula;
a handle surrounding the barrel and providing a reinforced location for a user to grasp the cannula; and
one or more reinforcing fibers positioned between an interior and an exterior of the barrel.

2. The device of claim 1 wherein the cannula is shaped and sized for fat harvesting or fat grafting.

3. The device of claim 1 wherein the cannula has an external diameter of about 2 mm to about 8 mm.

4. The device of claim 1 wherein the cannula has a length of about 5 cm to about 40 cm.

5. The device of claim 1 further comprising a fiber optic extending from a first end of the barrel to a second end of the barrel.

6. The device of claim 1 wherein the barrel is formed of a translucent material.

7. The device of claim 1 wherein the barrel is formed of a transparent material.

8. The device of claim 1 wherein the barrel is formed of at least one of a glass and a plastic.

9. The device of claim 1 wherein the barrel includes a stainless steel portion.

10. The device of claim 1 wherein the one or more reinforcing fibers are straight wires oriented linearly and axially along the barrel.

11. The device of claim 10 wherein the one or more reinforcing fibers are equally circumferentially spaced about the barrel.

12. The device of claim 1 wherein the one or more reinforcing fibers are arranged as a helical coil of two or more fibers.

13. The device of claim 12 wherein the one or more reinforcing fibers are equally circumferentially spaced about the barrel.

14. The device of claim 1 wherein the one or more reinforcing fibers including a reinforcing mesh.

15. The device of claim 1 wherein the one or more reinforcing fibers are formed of stainless steel.

16. The device of claim 1 wherein the device is an injection cannula shaped and sized for use in fat transplantation and having a gauge from 12 gauge to 16 gauge.

17. The device of claim 1 wherein the barrel includes one or more holes along its length to facilitate distribution of material during an injection procedure.

18. The device of claim 17 wherein the barrel includes five holes.

19. The device of claim 1 wherein the insertion tip includes a spatulated end.

20. The device of claim 1 wherein the insertion tip includes a sharpened leading edge.

21-40. (canceled)
